(19) United States
(12) Patent Application Publication
(10) Pub. No.: US 2009/0105578 A1
(43) Pub. Date: Apr. 23, 2009

(54) Interactive Medical Imaging Processing and User Interface System

(75) Inventor: Wei Qu, Schaumburg, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/203,371

(22) Filed: Sep. 3, 2008

Related U.S. Application Data
(60) Provisional application No. 60/981,222, filed on Oct. 19, 2007.

(51) Int. Cl. A61B 5/05

(52) U.S. Cl. 600/407

(57) ABSTRACT

An interactive medical user interface system presents a user interactive image window, including a distribution curve of an organ section area over a heart beat cycle time and supports a desired clinical workflow. An interactive medical image processing and user interface system for use in patient organ imaging includes an image data processor. The image data processor processes data representing multiple images of an organ of a patient over the heart beat cycle of the patient to derive data representing a distribution curve of an organ section area over a heart beat cycle time. A user interface generates data representing a composite user interface display image including, a first user interactive image window presenting the distribution curve and a second image window presenting an image of the organ corresponding to a location on the distribution curve interactively selected by a user via the first user interactive image window.

This is a non-provisional application of provisional application Ser. No. 60/981,222 filed Oct. 19, 2007, by W. Qu.

### FIELD OF THE INVENTION

This invention concerns an interactive medical image processing and user interface system for use in patient anatomical organ imaging involving presenting a user interactive image window including a distribution curve of an organ section area over a heart beat cycle time and an image of a patient organ.

### BACKGROUND OF THE INVENTION

Ventricular angiography is commonly employed in examining cardiac functions and determining heart parameters representing stroke volume, ejection fraction, and heart wall motions. Known systems involve determining an end-diastolic image frame (ED) and an end-systolic image frame (ES) from an angiographic image sequence for use in quantizing heart parameters. Known systems typically employ a workflow for ED and ES selection in left ventricular analysis as illustrated in FIG. 1. In the known clinical workflow an Angiographic image sequence is acquired 103 and browsed 105 to identify and select a cardiac cycle with good image contrast 107. Both ED and ES image frames are manually selected in steps 109 and 111 in response to browsing the image sequence and comparing left ventricle area change in adjacent image frames. A user carefully visually inspects the image sequence to locate ED and ES frames by comparing the variation of left ventricle area in adjacent image frames. This known process is time consuming, labor intensive and burdensome and average ED and ES selection time typically comprises more than 1 minute and involves 40 to 50 user selection commands for one patient imaging study. Many (e.g., hundreds) of left ventricular analyses may need to be performed daily in a catheterization department representing a substantial work burden involving difficult ventricular angiogram processing. A system according to invention principles addresses these deficiencies and related problems.

### SUMMARY OF THE INVENTION

An interactive medical image processing and user interface system presents a user interactive image window including a distribution curve of an organ section area over a heart beat cycle time and an image of a patient organ and supports a desired clinical workflow. An interactive medical image processing and user interface system for use in patient organ imaging includes an image data processor. The image data processor processes data representing multiple images of an organ of a patient over the heart beat cycle of the patient to derive data representing a distribution curve of an organ section area over a heart beat cycle time. A user interface generates data representing a composite user interface display image including, a first user interactive image window presenting the distribution curve and a second image window presenting an image of the organ corresponding to a location on the distribution curve interactively selected by a user via the first user interactive image window.

### BRIEF DESCRIPTION OF THE DRAWING

FIG. 1 illustrates a known workflow process involved in end-diastolic image frame (ED) and end-systolic image frame (ES) selection in left ventricular analysis.

FIG. 2 shows an interactive medical image processing and user interface system for use in patient organ imaging, according to invention principles.

FIG. 3 illustrates a clinical workflow employed by an interactive medical image processing and user interface system, according to invention principles.

FIG. 4 illustrates the operational relationship between interactive medical user interface image windows, according to invention principles.

FIGS. 5, 6 and 7 comprise different user interface image embodiments employed by an interactive medical image processing and user interface system, according to invention principles.

FIG. 8 shows a flowchart of a process performed by an interactive medical image processing and user interface system for use in patient organ imaging, according to invention principles.

### DETAILED DESCRIPTION OF THE INVENTION

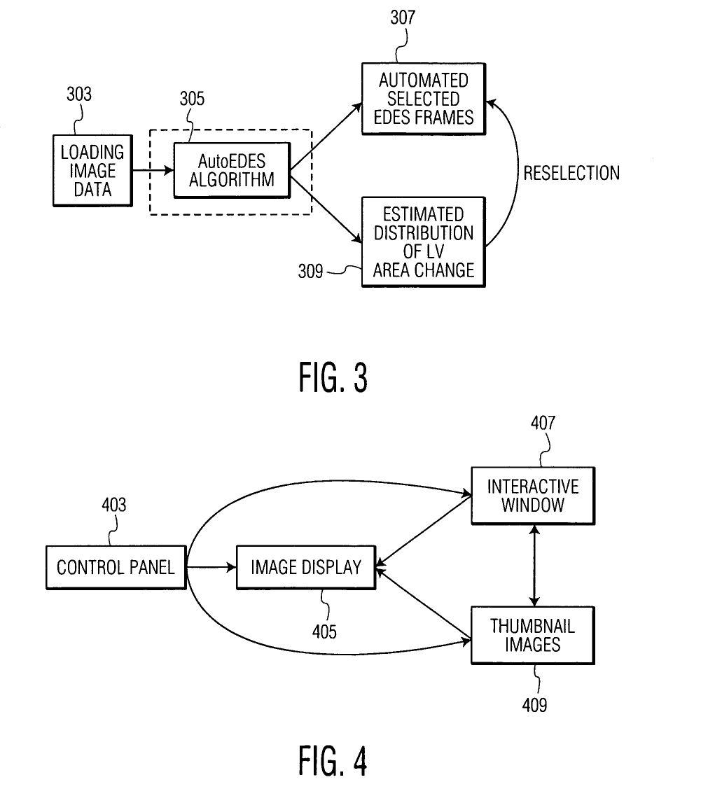

An interactive medical image processing and user interface system presents a user interactive image window including a distribution curve of an organ section area over a heart beat cycle time and an image of a patient organ and supports a desired clinical workflow. FIG. 3 illustrates a clinical workflow employed by the interactive medical image processing and user interface system in left ventricular analysis, for example. In the workflow process the system acquires and loads data representing multiple images of an organ of a patient in step 303 and in steps 305 and 307 automatically detects an end-diastolic image frame (ED) and end-systolic image frame (ES) using one of a variety of different known processes and provide frame numbers indicating the identified images. The ED and ES images are thereby subsequently accessible for display on a workstation. In step 309, the system also estimates a distribution of patient left ventricle area change over multiple heart cycles for display on the workstation. A user is advantageously able to examine the distribution over multiple cardiac cycles and use a displayed distribution curve to quickly localize and identify ED and ES images or other cardiac cycle images for presentation using the curve which intuitively presents the cardiac cycles in the curve as peaks and valleys. In another embodiment, the ED and ES image frames are manually determined in response to user image review and selection. The automatic ED and ES image detection is performed seamlessly and transparently in the background without user involvement.

A processor as used herein is a device for executing stored machine-readable instructions for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

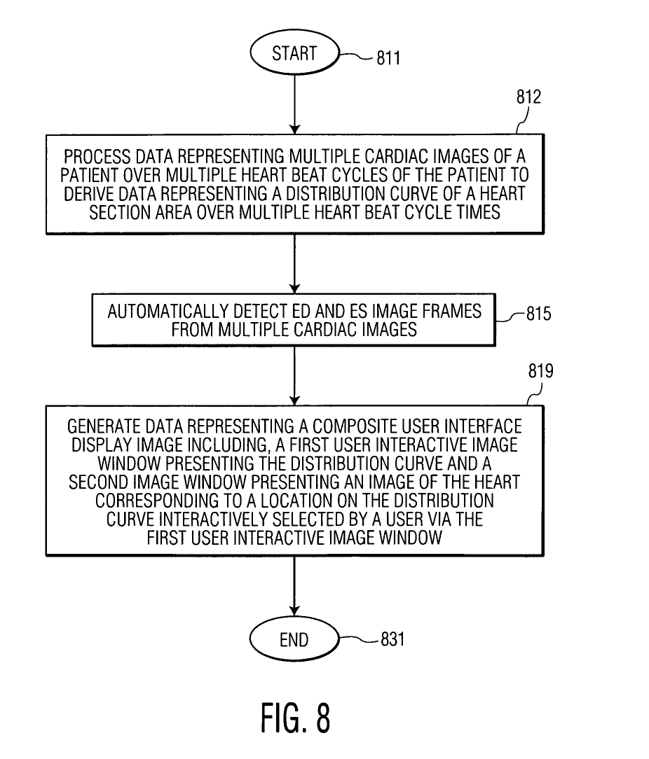

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps (e.g., of FIG. 8) herein may be performed automatically or wholly or partially in response to user command. An activity (including a step) performed automatically is performed in response to executable instruction or device operation without user direct initiation of the activity. Workflow comprises a sequence of tasks performed by a device or worker or both. An object or data object comprises a grouping of data, executable instructions or a combination of both or an executable procedure.

A workflow processor, as used herein, processes data to determine tasks to add to, or remove from, a task list or modifies tasks incorporated on, or for incorporation on, a task list. A task list is a list of tasks for performance by a worker or device or a combination of both. A workflow processor may or may not employ a workflow engine. A workflow engine, as used herein, is a processor executing in response to predetermined process definitions that implement processes responsive to events and event associated data. The workflow engine implements processes in sequence and/or concurrently, responsive to event associated data to determine tasks for performance by a device and or worker and for updating task lists of a device and a worker to include determined tasks. A process definition is definable by a user and comprises a sequence of process steps including one or more, of start, wait, decision and task allocation steps for performance by a device and or worker, for example. An event is an occurrence affecting operation of a process implemented using a process definition. The workflow engine includes a process definition function that allows users to define a process that is to be followed and includes an Event Monitor, which captures events occurring in a Healthcare Information System. A processor in the workflow engine tracks which processes are running, for which patients, and what step needs to be executed next, according to a process definition and includes a procedure for notifying clinicians of a task to be performed, through their worklists (task lists) and a procedure for allocating and assigning tasks to specific users or specific teams.

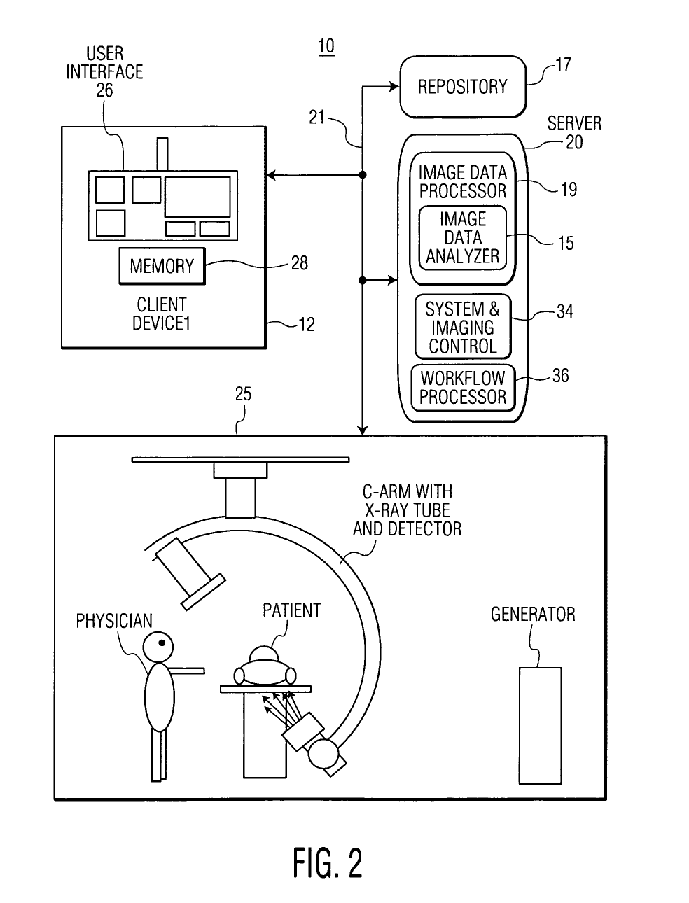

FIG. 2 shows an interactive medical image processing and user interface system 10 for use in patient organ imaging. System 10 includes one or more processing devices (e.g., workstation or portable device such as notebooks, Personal Digital Assistants, phones) 12 that individually include memory 28 and a user interface 26 supporting image presentation in response to user command and predetermined user (e.g., physician) specific preferences. System 10 also includes at least one repository 17, X-ray imaging modality system 25 (which in an alternative embodiment may comprise an MR (magnetic resonance), CT scan, or Ultra-sound system, for example) and server 20 intercommunicating via network 21. User interface 26 provides data representing display images comprising a Graphical User Interface (GUI) for presentation on processing device 12. At least one repository 17 stores medical image studies for multiple patients in DICOM compatible (or other) data format. A medical image study individually includes multiple image series of a patient anatomical portion which in turn individually include multiple images. Server 20 includes image data processor 19 including image data analyzer 15 and system and imaging controller 34 as well as workflow processor 36.

Image data processor 19 processes data representing multiple images of an organ of a patient over the heart beat cycle of the patient to derive data representing a distribution curve of an organ section area over a heart beat cycle time. Imaging system 10 acquires data representing multiple temporally sequential individual images of a patient organ using X-ray modality system 25. X-ray modality system 25 comprises a C-arm X-ray radiation source and detector device rotating about a patient table and an associated electrical generator for providing electrical power for the X-ray radiation system. User interface 26 generates data representing a composite user interface display image including, a first user interactive image window presenting the distribution curve and a second image window presenting an image of the organ corresponding to a location on the distribution curve interactively selected by a user via the first user interactive image window. The distribution curve indicates an end-diastolic (ED) location and end-systolic (ES) location and image data analyzer 15 automatically detects ED and ES image frames from multiple cardiac images. In response to user selection of the end-diastolic (ED) location or the end-systolic (ES) location, a corresponding ED or ES image frame is presented in the second image window. Workflow processor 36 manages task sequences involved in system 10 operation including detecting ED and ES image frames from multiple cardiac images and generating distribution curves.

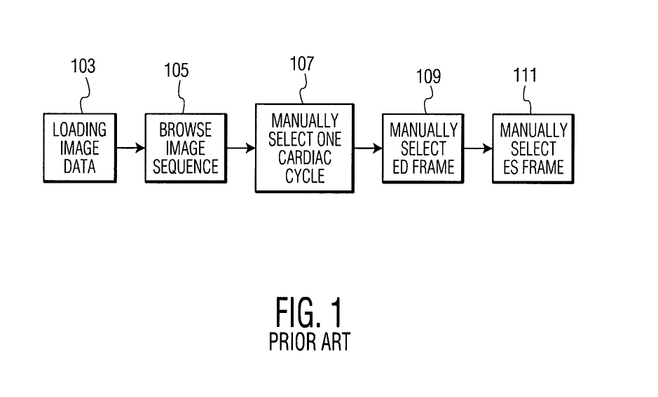

FIG. 4 illustrates the operational relationship between interactive medical user interface image windows provided by user interface 26 (FIG. 1). In response to a single user command, optimal ED and ES image frames (identified by an image frame number, for example) are automatically determined (by one of a number of known processes) by image data processor 19. Thus, the identified optimal ED and ES image frames may be accessed and presented without user browsing and reviewing of a whole image sequence. Further, in response to the single user command, user interface 26 provides an interactive composite image window including an estimated left ventricle area distribution curve together with a user selected ED or ES image frame (selected by user choice of a location on the distribution curve) for display on workstation 12. A user interactively selects a point inside an interactive popup image window in the composite image by using an arrow key to shift a selected point on the estimated left ventricle area distribution curve, for example. The left ventricle image corresponding to the selected point is concurrently presented on workstation 12. Thereby, a user may readily browse an image sequence by traversing the displayed left ventricle area distribution curve to quickly locate any desirable left ventricle image frame without sequentially looking through a whole image sequence. Further, neighboring images of automatically identified ED and ES image frames or manually identified ED and ES image frames via user selection of points on the distribution curve, are presented as thumbnail images. A user thereby is able to readily navigate through the thumbnail images and choose a desired image for display by selection of a corresponding thumbnail image. This facilitates user confirmation of correctness of automatically, or manually, selected ED and ES image frames by quick visual inspection.

FIG. 4 illustrates the operational relationship between interactive medical user interface image windows provided by user interface 26 (FIG. 1). A user is able to adjust parameters of an automatic ED and ES image selection process (e.g., an algorithm) via a displayed control panel image window in step 403. In response to change of parameters via the control panel image window, new estimated ED and ES images are selected via an interactive image window 407 and presented together with thumbnail (reduced size) medical images 409 by update of a composite image display on workstation 12 in step 405. A user is able to browse and adjust ED and ES image selection by using either interactive image window 407 or thumbnail images 409. In response to a change initiated via interactive image window 407, a corresponding change is substantially immediately reflected in thumbnail images 409.

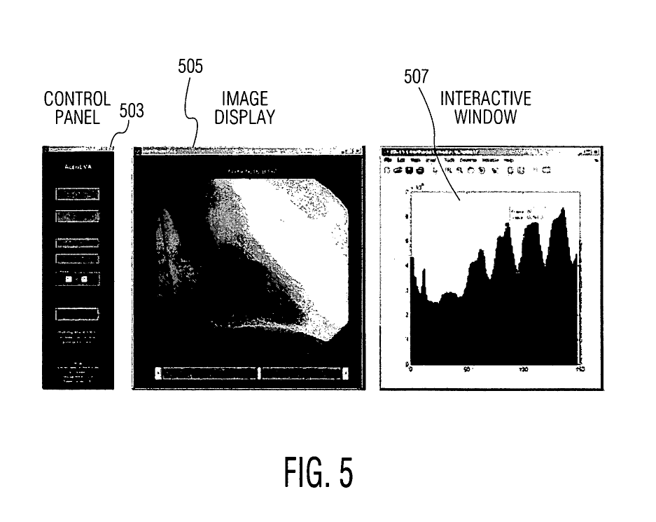

FIGS. 5, 6 and 7 comprise different user interface image embodiments employed by an interactive medical image processing and user interface system provided by user interface 26 (FIG. 1) for display on workstation 12. The composite display image includes control panel image window 503 enabling user adjustment of parameters of an automatic ED and ES image selection process, medical image display window 505 and interactive image window 507 showing estimated distribution of patient left ventricle area change.

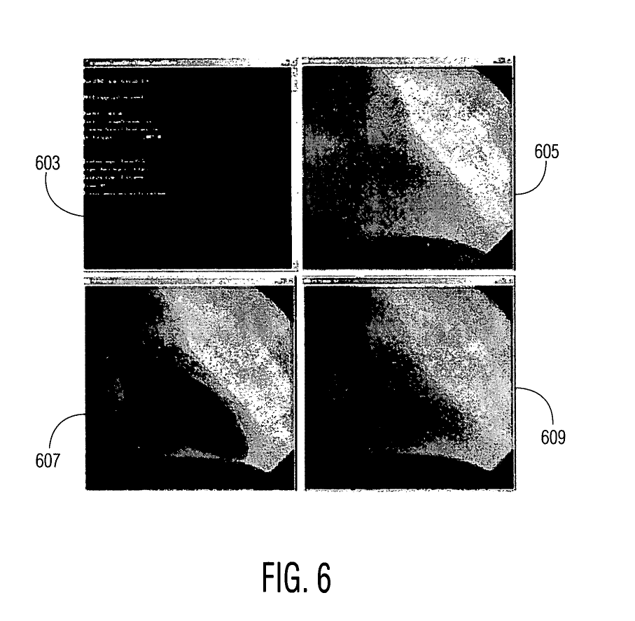

FIG. 6 illustrates a composite display image including four image windows, for example. Image window 603 comprises an information display window for presenting information associated with displayed medical images and image window 605 comprises a currently selected medical image data display window.

Image windows 607 and 609 present estimated ED and ES image frames respectively.

In another embodiment image window 603 or 605 may comprise a control panel image window or an interactive image window showing estimated distribution of patient left ventricle area change, for example.

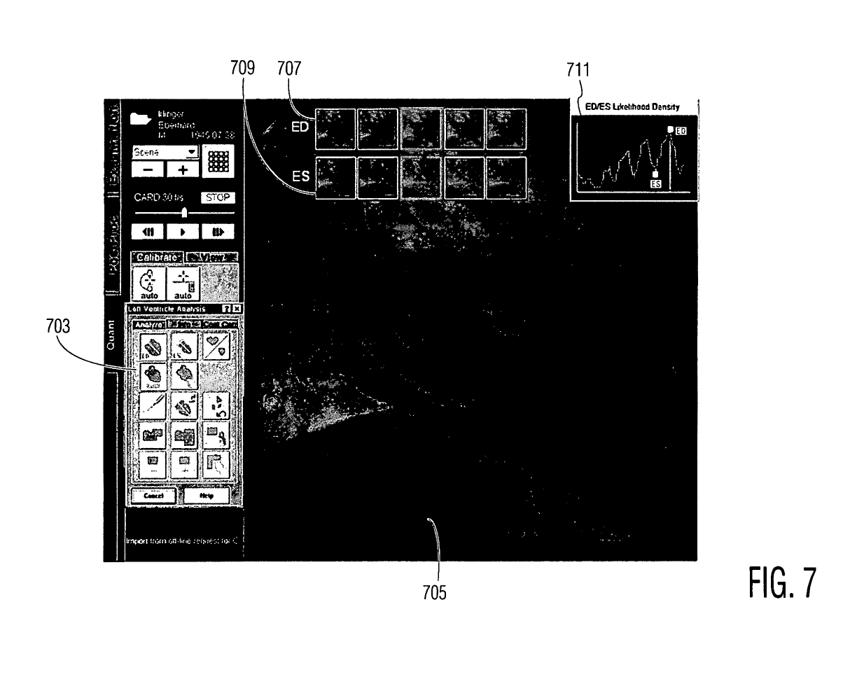

FIG. 7 illustrates a composite display image including control panel image window 703 enabling user adjustment of parameters of an automatic ED and ES image selection process and other imaging parameters. Interactive image window 711 shows an estimated distribution of patient left ventricle area change over multiple heart beat cycles and includes user selectable and movable cursor locations indicating ED and ES (or other) points on the distribution curve. Rows 707 and 709 of reduced size (thumbnail) images show sequences of five images with center images corresponding to the two movable cursor locations selected on the distribution curve 1 shown in interactive image window 711. Here the center images of rows 707 and 709 are reduced size images of the selected ED and ES points on the distribution curve of window 711 and rows 707 and 709 enable a user to quickly review reduced size images adjacent the selected ED and ES center images to see if the adjacent images are better candidates for selection as ED and ES images, for example. A reduced size image is displayed in full size in image window 705 in response to user selection of the reduced size image. Selection of a point on the distribution curve in window 711 also results in a corresponding medical image being displayed in window 705.

FIG. 8 shows a flowchart of a process performed by interactive medical image processing and user interface system 10 for use in patient organ imaging. In step 812, following the start at step 811, image data processor 19 processes data representing multiple cardiac images (or organ images) of a patient over multiple heart beat cycles of the patient to derive data representing a distribution curve of a heart (or organ) section area over a plurality of heart beat cycle times. The cardiac images (or organ images) comprise at least one of, (a) X-ray 2D images, (b) MR images, (c) Ultrasound images and (d) CT scan images. Also, the heart section area comprises at least one of, (a) a Left Ventricle area and (b) a Right Ventricle area.

The distribution curve indicates an end-diastolic (ED) location and end-systolic (ES) location, for a heart for example. In step 815, image data analyzer 15 automatically detects ED and ES image frames from multiple cardiac images. Workflow processor 36 manages a task sequence including detecting ED and ES image frames from multiple cardiac images and generating the distribution curve.

In step 819, user interface 26 generates data representing a composite user interface display image including, a first user interactive image window presenting the distribution curve and a second image window presenting an image of the heart (or organ) corresponding to a location on the distribution curve interactively selected by a user via the first user interactive image window. In response to user selection of the end-diastolic (ED) location or the end-systolic (ES) location, a corresponding ED or ES image frame is presented in the second image window. The composite user interface display image includes multiple reduced size sequential cardiac images indicating ED and ES images in the sequence and enables a user to scroll through the multiple reduced size sequential cardiac images in response to user image element selection.

Image data processor 19 automatically derives data representing the distribution curve of the heart cardiac images (or organ) section area over the heart beat cycle time by determination of a boundary of the heart (or organ) section area in different images over the heart beat cycle time and computation of an area within the boundary. Image data processor 19 recognizes the boundary based on image luminance variation in response to predetermined cardiac element recognition rules. In another embodiment the boundary is recognized based on image luminance variation in response to user command. The process of FIG. 8 terminates at step 831.

The systems and processes of FIGS. 2-8 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The interactive medical image processing and user interface system is usable to provide a user interactive image window including a distribution curve of an organ area over a heart beat cycle time and enabling a user to initiate generation of data associated with a user selected location on the distribution curve. The processes and applications may, in alternative embodiments, be located on one or more (e.g., distributed) processing devices accessing a network linking the elements of FIG. 2. Further, any of the functions and steps provided in FIGS. 2-8 may be implemented in hardware, software or a combination of both and may reside on one or more processing devices located at any location of a network linking the elements of FIG. 2 or another linked network, including the Internet.

What is claimed is:

1. An interactive medical image processing and user interface system for use in patient organ imaging, comprising:
an image data processor for processing data representing a plurality of images of an organ of a patient over the heart beat cycle of said patient to derive data representing a distribution curve of an organ section area over a heart beat cycle time; and
a user interface for generating data representing a composite user 1interface display image including,
a first user interactive image window presenting said distribution curve and
a second image window presenting an image of said organ corresponding to a location on said distribution curve interactively selected by a user via said first user interactive image window.

2. A system according to claim 1, wherein
said organ comprises a heart,
said image data processor processes data representing a plurality of cardiac images of a patient over a plurality of heart beat cycles to derive data representing a distribution curve of a heart section area over a plurality of heart beat cycle times.

3. A system according to claim 2, wherein
said heart section area comprises at least one of, (a) a Left Ventricle area and (b) a Right Ventricle area.

4. A system according to claim 1, wherein
said plurality of organ images comprises at least one of, (a) X-ray 2D images, (b) MR images, (c) Ultrasound images and (d) CT scan images

5. A system according to claim 1, wherein
said image data processor automatically derives data representing said distribution curve of said organ section area over said heart beat cycle time by determination of a boundary of said organ section area in different images over said heart beat cycle time and computation of an area within said boundary, said boundary being recognized based on image luminance variation in response to predetermined cardiac element recognition rules.

6. A system according to claim 1, wherein
said image data processor derives data representing said distribution curve of said organ section area over said heart beat cycle time by determination of a boundary of said organ section area in different images over said heart beat cycle time and computation of an area within said boundary, said boundary being recognized based on image luminance variation in response to user command.

7. An interactive medical image processing and user interface system for use in patient organ imaging, comprising:
an image data processor for processing data representing a plurality of cardiac images of a patient over a plurality of heart beat cycles of said patient to derive data representing a distribution curve of a heart section area over a plurality of heart beat cycle times; and
a user interface for generating data representing a composite user interface display image including,
a first user interactive image window presenting said distribution curve and
a second image window presenting an image of said heart corresponding to a location on said distribution curve interactively selected by a user via said first user interactive image window.

8. A system according to claim 7, wherein
said distribution curve indicates an end-diastolic (ED) location and end-systolic (ES) location.

9. A system according to claim 8, wherein
in response to user selection of said end-diastolic (ED) location or said end-systolic (ES) location, a corresponding ED or ES image frame is presented in said second image window.

10. A system according to claim 8, including
an image data analyzer for automatically detecting ED and ES image frames from a plurality of cardiac images.

11. A system according to claim 10, including
a workflow processor for managing a task sequence including detecting ED and ES image frames from a plurality of cardiac images and generating said distribution curve.

12. A system according to claim 7, wherein
said composite user interface display image includes a plurality of reduced size sequential cardiac images indicating ED and ES images in the sequence.

13. A system according to claim 12, wherein
said composite user interface display enables a user to scroll through said plurality of reduced size sequential cardiac images in response to user image element selection.

14. A system according to claim 7, wherein
said image data processor automatically derives data representing said distribution curve of said heart section area over said heart beat cycle time by determination of a boundary of said heart section area in different images over said heart beat cycle time and computation of an area within said boundary, said boundary being recognized based on image luminance variation in response to predetermined cardiac element recognition rules.

15. A system according to claim 7, wherein
said image data processor derives data representing said distribution curve of said heart section area over said heart beat cycle time by determination of a boundary of said heart section area in different images over said heart beat cycle time and computation of an area within said boundary, said boundary being recognized based on image luminance variation in response to user command.
